(19) United States
(12) Patent Application Publication
(10) Pub. No.: US 2002/0127277 A1
(43) Pub. Date: Sep. 12, 2002

(54) Solid dosage forms of divalproex sodium

(76) Inventors: Yihong Qiu, Gurnee, IL (US); Kevin R. Engh, Kenosha, WI (US); Lynn Faitsch, Libertyville, IL (US); Russell T. Slade, Lindenhurst, IL (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 09/748,659

(22) Filed: Dec. 22, 2000

(51) Int. Cl. A61K 9/54; A61K 9/14

(52) U.S. Cl. 424/489; 424/458

(57) ABSTRACT

The present invention is directed to an aqueous process for granulating valproate compounds, in which the pH of the granulation solution is maintained at a pH of 5 or below. The invention is also directed to dosage forms in which the residual content of organic solvents is reduced to a level of 0.2 w/w % or less.

The present invention is directed to new solid dosage forms of divalproex sodium, valproic acid, prodrugs of valproic acid, and analogs of valproic acid (hereinafter “valproate compounds”). Other aspects of the invention are directed to improved methods of producing these new dosage forms and to improved methods of granulating these valproate compounds.

### BACKGROUND

Compressed tablets are the most common means of administering drugs. These dosage forms have a number of advantages. They are typically less expensive to produce. They are relatively small and thus are easy for the patient to swallow. For pediatric indications, they can be formulated as chewable tablets.

The initial starting material for any tablet is the bulk drug substance (i.e. the drug). This bulk drug substance is typically present as a powder. This powder cannot be compressed directly into a tablet. It lacks sufficient binding capacity to form an agglomerate. Further, it lacks the lubricating and flowing characteristics that are also necessary in tablet manufacture.

Typically, the initial step in the manufacture of any tablet is a granulation. Granulation is a process of size enlargement, whereby small particles of the bulk drug are gathered together into larger aggregates capable of being compressed into solid dosage forms. This is accomplished by adding binders to the admixture during the granulation process. The binder helps to increase the adhesiveness of the particles and thereby facilitate the size enlargement process.

There are a variety of granulation techniques known in the art. The most common is a wet granulation. Wet granulation is well known in the art and is described in detail in works such as PHARMACEUTICAL DOSAGE FORMS, Tablets, Lieberman et al., Marcel Dekker, Inc. pages 148-149. Other descriptions may be found in Remington's Pharmaceutical Sciences, Fifteenth Edition pages 1583-1586 (1975).

In a wet granulation process, the bulk drug is contacted with both a solvent and a binder. A minimal quantity of solvent is utilized. The volume of solvent should be such as to produce a mass having an appearance similar to dough. The quantity of solvent should not be sufficient to actually dissolve most of the drug substance, particularly when drug loading is high, as is the case for divalproex sodium. The wetted mass is then mixed in the presence of the binder to produce the agglomerated particles of bulk drug that are required for tablet compression. The function of the solvent is to facilitate the interaction between the binder and the particles of drug substance that is associated with granulation.

A variety of solvents have been used as granulation aids. Typical solvents include water, alcohols such as methanol, ethanol, propanol, etc., and organic solvents such as methylene chloride, acetone, butanone, etc. Water is the most preferred granulating solvent. It is non-toxic, has absolutely no adverse consequences on the environment, and is inexpensive. It also is safer for the workers producing the tablets since it has no potential to burn or explode. The only disadvantage of water is that it is not suitable for all applications.

Water is not a suitable granulating aid for formulations in which a high drug loading factor is desired (e.g. >60%) and in which the drug is very water soluble. In such applications the water dissolves the compound, rather than facilitating the interaction between the particles of drug and binder that occur during agglomeration. Therefore, alcohols or organic solvents are used to agglomerate such formulations. While such solvents do not have any negative effects on the granulation process, they do suffer from practical disadvantages.

Organic solvents such as methylene chloride have negative consequences on the environment. Special precautions must be taken in their disposal to avoid environmental contamination. Further, organic solvents can present safety hazards to the employees during the manufacturing of such formulations. These solvents are explosive and flammable. Incidents have been reported in which workers have been injured from accidental fires. Organic solvents and alcohols are considerably more expensive than water. This plus the difficulty of disposal adds cost to the production process and ultimately to the consumer of the medication. Further, it is impossible to completely remove all traces of the organic solvent or alcohol from the granulated drug substance. Trace solvents can leave adverse odors in the finished dosage form that are objectionable to many patients.

Valproic acid has a long history of use as a medication. It and other valproate compounds have been used in the treatment of neurological conditions such as epilepsy, migraine, and mania. As its name implies, valproic acid contains a carboxylic acid function. This makes its salt extremely hydrophilic (i.e. water soluable).

A number of dosage forms containing valproic acid, and other valproate compounds, have been described in the literature. U.S. Pat. No. 5,019,398 describes a sustained release formulation of valproic acid having efficacy in the treatment of epilepsy. The '398 patent describes a tablet. The initial step in its manufacture is a granulation. The '398 patent specifies that the valproate should be granulated with an organic solvent such as alcohols, ketones or halogenated hydrocarbons. The most preferred granulating solvent is ethanol (see page 3, 4^{th} paragraph of Web version).

Similar disclosures may be found in a review of the literature for valproic acid and other valproate compounds. U.S. Pat. No. 5,598,191 shows the preparation of sodium valproate tablets. Examples 1, and 3-6 utilize a granulation step. Ethanol is used as the granulating solvent in all of these examples. U.S. Pat. No. 5,268,182 discloses the preparation of sustained release tablets containing divalproex sodium. Examples 1 and 2 show the preparation of a divalproex sodium dosage form in which ethanol was used as the granulation solvent. U.S. Pat. No. 5,049,586 discloses the preparation of moisture stable valproic tablets. In example I, it shows ethanol being using as the granulation solvent.

Thus conventional wisdom in the prior art teaches that organic solvents must be used in any granulation process involving valproic acid and other valproate compounds. Thus a need exists in the art for a granulation process that may be used to produce valproate dosage forms that do not require an organic solvent. It would be a significant advance in the art to develop an aqueous process for granulating valproate compounds.

### SUMMARY OF THE INVENTION

It has been discovered that aqueous granulations may be carried out with valproic acid, divalproex sodium and other valproate compounds, provided the pH of the aqueous solution is maintained at a level of 5 or below. Valproate has limited solubility at such a pH range. Aqueous solutions within this range will not dissolve significant quantities of the valproate compound. Instead, the aqueous solution will facilitate the interactions between the drug particles, diluents, and binders that are associated with the agglomeration that occurs in granulation. The granulation is carried out in the same manner as those described in the prior art above. The only modification is that an aqueous solution containing a food grade acid is used as the granulating solvent.

As described above, the aqueous granulation of this invention has numerous advantages when compared with the organic granulations of the prior art. This process does not have the environmental and safety drawbacks of the prior art. Further, costs are significantly reduced with this process. The pharmaceutical dosage forms produced via the aqueous granulation also exhibit numerous advantages when compared with the prior art dosage forms.

Granulations carried out with organic solvents inherently result in residual amounts of the organic solvent being retained in the granulate. The tablets and capsules produced from this granulate will also contain comparable levels of the organic solvent. These residual levels of organic solvents are typically present in sufficient concentrations to be detected by the human olfactory glands. Patients will complain about the smell and off-taste of the dosage form. While the patient assumes it is the medicine that they are smelling, it is actually the residual organic solvent that is being detected.

For example, Abbott Laboratories markets a valproate compound by the name of Depakote®. It's active constituent is divalproex sodium. Divalproex sodium is formulated using a process in which ethanol is the granulating solvent. Chemical analysis of Depakote tablets typically detects the presence of ethanol in the quantity of 0.7-1.2 w/w % based upon the weight of the final tablets. This level is well within the detection limits of the human olfactory gland.

By contrast, dosage forms produced using the aqueous process described above will not have such significant levels of organic solvents. Dosage forms produced using this process will contain at most 0.1-0.2 w/w % of an organic solvent (based upon the weight of the final tablets) and more typically will contain levels below the limit of detection when measured by static headspace gas chromatgraphy. Such levels will not produce the off aroma's or bad tastes associated with the valproic dosage forms of the prior art.

### DETAILED DESCRIPTION OF THE INVENTION

As noted above, the invention relates to new and improved dosage forms of valproic acid and other valproate compounds. Further aspects of the invention also relate to methods for producing these improved dosage forms. Several valproate compounds are currently available commercially in the United States or have been described in the literature.

One such compound is valproic acid. Valproic acid may be represented by the following structure:

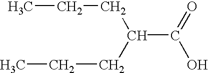

Valproic acid is available commercially from Abbott Laboratories of Abbott Park, Ill. Methods for its synthesis are described in Oberreit, Ber. 29, 1998 (1896) and Keil, Z. Physiol. chem. 282, 137 (1947). It's activity as an antiepileptic compound is described in the Physician Desk Reference, 52nd Edition, page 421, 1998.

The sodium salt of valproate is also known in the art as an anti-epileptic agent. It is also known as sodium valproate and is described in detail in The Merck Index, 12 Edition, page 1691, (1996). Further descriptions may be found in the Physician Desk Reference, 52nd Edition, page 417, 1998.

Divalproex sodium is effective as an antiepileptic agent, in the treatment of migraine and for bipolar disorders. Methods for its preparation may be found in U.S. Pat. Nos. 4,988,731 and 5,212,326, the contents of both which are hereby incorporated by reference.

In addition to these specific compounds, one of ordinary skill in the art would readily recognize that the carboxylic moiety of the valproic compound may be functionalized in a variety of ways. This includes forming compounds which readily metabolize in-vivo to produce valproate, such as valproate amide (valproimide), as well as other pharmaceutically acceptable amides and esters of the acid (i.e. prodrugs). This also includes forming a variety of pharmaceutically acceptable salts.

Suitable pharmaceutically acceptable basic addition salts include, but are not limited to cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Other possible compounds include pharmacuetically acceptable amides and esters. “Pharmaceutically acceptable ester” refers to those esters which retain, upon hydrolysis of the ester bond, the biological effectiveness and properties of the carboxylic acid and are not biologically or otherwise undesirable. For a description of pharmaceutically acceptable esters as prodrugs, see Bundgaard, E., ed., (1985) Design of Prodrugs, Elsevier Science Publishers, Amsterdam, which is hereby incorporated by reference. These esters are typically formed from the corresponding carboxylic acid and an alcohol. Generally, ester formation can be accomplished via conventional synthetic techniques. (See, e.g., March Advanced Organic Chemistry, 3rd Ed.,John Wiley & Sons, New York (1985) p. 1157 and references cited therein, and Mark et al. Encyclopedia of Chemical Technology,John Wiley & Sons, New York (1980), both of which are hereby incorporated by reference. The alcohol component of the ester will generally comprise (i) a C₂-C₁₂ aliphatic alcohol that can or can not contain one or more double bonds and can or can not contain branched carbons or (ii) a C₇-C₁₂ aromatic or heteroaromatic alcohols. This invention also contemplates the use of those compositions which are both esters as described herein and at the same time are the pharmaceutically acceptable salts thereof.

“Pharmaceutically acceptable amide” refers to those amides which retain, upon hydrolysis of the amide bond, the biological effectiveness and properties of the carboxylic acid and are not biologically or otherwise undesirable. For a description of pharmaceutically acceptable amides as prodrugs, see Bundgaard, H., ed., (1985) Design of Prodrugs, Elsevier Science Publishers, Amsterdam. These amides are typically formed from the corresponding carboxylic acid and an amine. Generally, amide formation can be accomplished via conventional synthetic techniques. (See, e.g., March Advanced Organic Chemistry, 3rd Ed., John Wiley & Sons, New York (1985) p. 1152 and Mark et al. Encyclopedia of Chemical Technology, John Wiley & Sons, New York (1980)., both of which are hereby incorporated by reference. This invention also contemplates the use of those compositions which are both amides as described herein and at the same time are the pharmaceutically acceptable salts thereof.

Analogs of valproate have also been described in the literature. For example, biological activity has been retained in compounds in which one of the propyl chains have been eliminated from the molecule. One of these entities is known as isovaleramide. It's structure and activity are described in U.S. Pat. Nos. 5,763,494 and 5,506,268, the contents of both which are hereby incorporated by reference.

Other analogs have been described in U.S. patent application Ser. No. 09/258,882, filed Mar. 1, 1999, the contents of which are hereby incorporated by reference. These structures may be represented by the following formula:

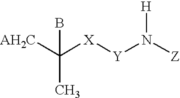

Where:

A=H, CH₃ or OH,

B=H, OH, or CH₃

X=CH₂, CHCH₃, C(CH₃)₂, —O—, CH(OH), or —CH₂O—,

Y=—CO—, or —SO₂—, and

Z=H, CH₂CO₂H, or CH₂CONH₂

Isovaleramide may be represented by the structure above in which Z is H, Y is CO, X is CH₂ and both B and A are H.

Other valproic analogs have been described in U.S. Pat. No. 5,585,358, the contents of which are hereby incorporated by reference. These valproic analogs may be represented by the following formula:

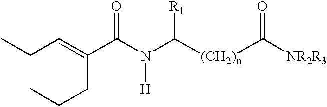

wherein R₁, R₂, and R₃ are independently the same or different and are hydrogen, C₁-C₆ alkyl, aralkyl, or aryl, and n is an integer which is greater than or equal to 0 and less than or equal to 3.

Further valproic analogs have been prepared in which either unsaturation or a cycloalkyl moiety has been incorporated into one of the proply mieties of the valproate structure. These compounds are described in U.S. Pat. No. 5,786,380, the contents of which are hereby incorporated by reference. The more preferred compounds of this patent may be represented by the structures below:

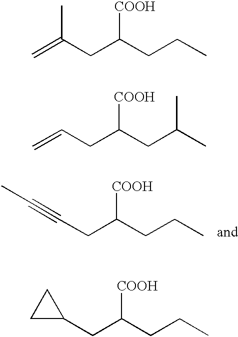

As used in this application, any reference to “valproate compounds” should be construed as including valproic acid, the sodium salt of valproate, divalproex sodium, any of the various salts of valproate described above, any of the prodrugs of valproate described above, and any of the analogs of valproate described above. Divalproex sodium is the most preferred valproate compound of the present invention.

As noted above, one aspect of the invention is directed to an improved process for granulating valproate compounds. The resulting product from that process is a granulate. As used in this application, the term “granulate” refers to valproate compounds that have been mixed with a binding agent sufficiently to produce granules that are suitable for compressing into tablets. The terms “granulate” and “agglomerate” should be considered as synonyms with this application.

A further aspect of this invention is an improved process for manufacturing solid dosage forms of valproate compounds. Typically, these dosage forms will be tablets. However, the valproic granulates produced by this invention may be incorporated into capsules, lozenges, chew able tablets, sprinkles, particles, etc. As used in this application, the term “solid dosage form” should be construed as covering any non-liquid pharmaceutical composition containing the valproic granulate of this invention, in a form suitable for oral consumption by a human being.

I. Overview of Process

The improved process of this invention is represented below in Scheme I.

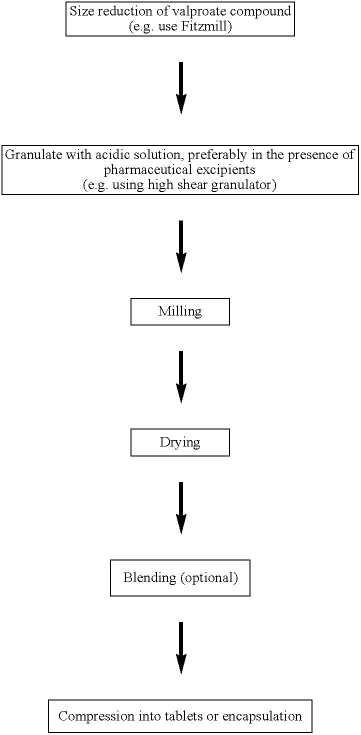

### Step A) Sizing

The starting material in the process is the drug substance (i.e. the valproate like compound). This drug substance will be in the form of a bulk powder. Typically, the initial step in the process is to subject the powdered valproic compound to a milling or screening step. This is done in order to reduce the particle size of the valproate compound. Many techniques are known in the art for accomplishing such a result. They are described in detail in Remington's Pharmaceutical Sciences, Fifteenth Edition, pages 1584-1585, (1975). Small batches can be forced by hand through a manual screen. Large quantities can be forced through sieving devices such as a Stokes oscillator, a Colton rotary granulator, a Stokes mill, a Fitzpatrick mill. etc. The valproic compound should be milled to a particle size of less than 4760 μm, and more preferably of less than 210 μm.

### Step B) Granulation

The next step of the process is the granulation step (wet granulation). It is carried out using techniques well known to those skilled in the pharmaceutical arts. Typically though, the valproate compound is admixed with one or more pharmaceutical excipients prior to carrying out the aqueous granulation. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, disintegrators, binders, etc. Such excipients are routinely used in the dosage forms of this invention.

Diluents are added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc. If a chewable tablet is desired, then mannitol, lactose, sorbitol, or inositol will be utilized.

Lubricants are incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc.

Glidant's are also typically incorporated into the formulation. A glidant improves the flow characteristics of the granulation. Examples of suitable glidant's include talc, silicon dioxide, and cornstarch.

Disintegrants are also typically incorporated into the formulations. They facilitate the break-up or disintegration of the tablet after it has been ingested by the patient. Examples of suitable disintegrating agents include starch and starch compounds such as, sodium starch glycolate, celluloses and cellulosic compounds, cross-linked polyvinylpyrrolidone, agar, bentonite, guar gum, microcrystalline cellulose, pregelatinized corn starch, croscarmalose, etc.

Binders are also incorporated into the formulation. Binders serve an important role in the granulation process. They facilitate the interaction between the individual powdered particles that are incorporated into the formulation (including the valproate compound and any excipient). This interaction allows the individual particles to adhere to each other so that they form an agglomerate. It is necessary for agglomeration to occur if the powdered mass is to be compressed into tablet. Examples of suitable binders include povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, antioxidants, or any other excipient commonly used in the pharmaceutical industry, etc.

The excipients and valproate compound are admixed together using techniques well known in the art. Typically, they will be dry blended together in a device such as a high shear mixer. Other suitable devices include V-blender, bin blender, etc.

The quantity of each individual excipient that is blended with the valproate compound will vary widely. However, they will be used in amounts that are typical for oral dosage forms. As a general guideline, the valproate compound will be present in an amount ranging from about 50-80 w/w % and the total quantity of excipients will be present in an amount ranging from about 20-50 w/w % (active and excipient each based upon the total tablet weight). More preferably, the valproate compound will be present in an amount ranging from about 60-75 w/w % and the total quantity of excipients will be present in an amount ranging from 25-40 w/w %. This relative ratio of excipent to valproate compound will also correspond to that present in the final dosage form. The water utilized in the granulation will be removed in the drying step. The quantity of food grade acid that is utilized during the granulation, which is described in greater detail below, is present in such minor amounts as to not alter this ratio.

Table I below provides guidelines for the quantities of excipients that may be utilized for the formulations. The specific amount of each of the various types of excipients can vary widely within these guidelines.

TABLE I
Quantity Guidelines (w/w % based upon total formulation wt)

| Substance | Typical | Preferred |
|---|---|---|
| Valproate compounds | 50-80% | 60-75% |
| Glidant | 0-6% | 2-5% |
| Diluent | 5-48% | 15-35% |
| Disintegrant | 0-10% | 3-5% |
| Lubricants | 0-5% | 0.5-1.5% |
| Binders | 2-10% | 3-4% |

The granulation is carried out using methods typically employed for wet granulations. The powdered admixture of excipients and valproate compound is placed in a container and contacted with an acidic solution (including a binder) under agitation. The container, or device, the granulation is carried out in is not critical. Any container capable of agitating the admixture may be utilized. A variety of such devices exist and are well known to those skilled in the art. Examples of suitable granulation devices include high shear mixers, low shear mixers, mixing tanks, etc. The rate or speed at which the valproic admixture is agitated, mixed or stirred can vary widely. As a general guideline, the rate of agitation is decreased as the volume of granulate increases. A speed of about 430-650 rpm is suitable for a volume of 10 liters, a speed of about 170-300 rpm is suitable for a volume of 75 liters, and a speed of about 120-185 rpm is suitable for a volume of 300 liters.

The composition of the acidic solution is not critical. It is preferably an admixture of water and a food grade acid. Any acid suitable for human consumption may be utilized. Examples of suitable acids include hydrochloric, phosphoric, maleic, acetic, citric, tartaric, and succinic, etc. If desired, minor quantities of an organic solvent may be introduced into the acidic solution. Solutions in which the organic content is kept below about 30 v/v % will not explode or burn and thus are relatively safe for workers to handle. However, admixtures containing organic solvents will present the environmental drawbacks discussed in detail above. As used in this application, the term “minor amounts of organic solvents” refers to a quantity of less than about 30 v/v % and more preferably less than about 10 v/v %.

The aqueous solution must have a pH of 5 or below. Utilizing solutions with a higher pH will cause the valproate compound to be dissolved rather than granulated. More preferably, a solution having a pH of 3 or less will be utilized. Most preferably, the solution will have a pH of 2 or less.

Producing solutions in such a pH range is well known to those skilled in the art. A 0.00001-0.1 normal (N) solution of a food grade acid will accomplish such a result. Examples of such solutions include a 0.01-0.04 N solution of citric acid, or a 0.001-0.1 N solution of hydrochloric acid.

The quantity of acidic solution that is utilized in the granulation process can vary widely. A sufficient quantity is utilized to produce a wetted mass resembling dough. This allows the agglomeration process to occur in which the individual particles adhere to one another. As a guideline, the acidic solution should be used in an amount equal to about 5-25 w/w %, based upon the total mass of excipients and valproate compound to be incorporated into the formulation. More preferably, the acidic solution is utilized in an amount equal to about 8-15 w/w %, as described above.

### Step C) Drying

The wetted mass produced in Step B must be dried so that it may be further processed. Typically, it will be dried to a moisture content of 3 w/w % or less, and more preferably 2 w/w %, or less. The material may be dried by placing it in an oven at a temperature range of from about 50-80° C. for a period time ranging from about 4-10 hours. Alternatively, it may be dried in a fluid bed dryer in which the mass is suspended and agitated in a warm air stream. These techniques are described in detail Remington's Pharmaceutical Sciences, supra, at pages 1585-1586, the disclosure of which is hereby incorporated by reference.

### Step D) Milling

Once the drying is completed, the granulate is milled to produce particles suitable for compression into tablets or encapsulation. This can be accomplished using the same techniques as described in Step A. A particle size in the range of about 44 μm-1200 μm should be obtained.

### Step E) Optional Blending

After the milling has been accomplished, additional pharmaceutical excipients may be introduced into the granulate for incorporation into the finished dosage form as described above. Such a step is not required and can vary depending upon the properties desired for the final dosage form. The quantity of any excipient added should be within the guidelines described above. Typically though, a glidant is added at this step of the process. Other excipients that may be incorporated at this step include lubricants, disintegrants, etc.

Any additional excipients can be introduced into the granulate using techniques well known to those skilled in the art. Typically they will be introduced via dry blending. Suitable dry blending devices include V-blenders, bin blenders, etc.

### Step F) Preparation of the Dosage Form

The final step of the process is the production of the solid dosage form. Typically, the granulate will be compressed directly into tablets. However, the granulate may also be filled into capsules, saches, lozenges, etc.

Methods for producing these solid dosage forms are well known to those skilled in the art. Methods for producing tablets are described in Remington's, supra at pages 1576-1598, the disclosure of which is hereby incorporated by reference. As a general guideline, a predetermined quantity of the granulate is placed in a steel cavity (lower punch). An upper punch is lowered into the steel cavity exerting pressure on the granulate thereby forming the tablet. The pressure required to form the tablet can vary widely depending upon the punch utilized, the desired tablet hardness, dissolution rate, etc. However as a general guideline a pressure in the range of about 10-36 kilo newtons (KN) will be utilized. Examples of suitable tablet punches include oval, concave, round, etc.

If desired, the tablets can be coated as is known in the art. Tablets are coated to improve their appearance, protect them from atmospheric degradation, control the site of drug release (i.e. enteric coatings), delay or prolong their release patterns, etc. Methods for coating tablets are well know in the art and have been described in detail by Remington's at pages 1608-1617 supra. The quantity of such coating can vary widely depending upon the reason for the coating. One skilled in the art can readily determine how much coating should be applied to the tablets in order to produce a desired result.

The granulation techniques described above are suitable for immediate release or delayed release tablets. As is know to those skilled in the art, polymers are incorporated into formulations to delay or extend their release patterns. One skilled in the art could adjust the granulation process described above to produce such delayed or extend release dosage forms.

In addition to tablets, the granulate can also be incorporated into capsules. Method for producing capsules are also well known in the art. Such methods are described in detail by Remington's supra at pages 1598-1605, the disclosure of which is hereby incorporated by reference. As a general principle, the granulate is prepared as described above, and then filled into gelatin capsules as is known in the art. A variety of automatated capsule filling machines are available to accomplish this result. Examples of suitable devices include Zanasi, MG-2, and Hoeflinger & Karg models.

II. New Dosage Forms

As has been previously described earlier in this application, one advantage of this invention is that it allows the production of solid dosage forms of valproate compounds in which the content of organic solvent is below that which can be detected by the human sense of smell or taste. The granulations of the prior art utilize organic solvents. Residual amounts of these organic solvents are retained in the granulate despite the attempts to remove such solvents during the drying step. The dosage forms produced from these granulates contain sufficient quantities of the organic solvent to produce an odor that is considered unpleasant by many consumers.

The human olfactory gland is surprisingly sensitive to the presence of organic solvents. A typical human can detect the presence of organic solvents in relatively minute amounts. For example, humans can detect ethanol by smell at a level of 100 parts per million in an aqueous solution, or acetone at a concentration of 500 part per million in an aqueous solution, ethylacetate of a level of 5 parts per billion, etc. The values at which humans detect common organic solvents, by both taste and smell, has been studied in detail. These values have been compiled on the internet at www.leffingwell.com/odorthre.htm, the contents of which are hereby incorporated by reference.

The solid dosage forms produced via this invention will have an organic solvent content of less than 0.2 w/w %, more preferably less than 0.1 w/w %, and most preferably less than the limit of detection when measured by static headspace gas chromatography. As used in this application, any reference to an organic solvent quantity refers to a level determined by static headspace gas chromatography using the methods of the European Pharmacopoeia, Supplement (2.2.28), page 12 (2001) as exemplified in Example 4.

As used in this application, the term organic solvent refers to a solvent containing hydrogen and carbon, other than water, and which is suitable for granulating pharmaceutical substances that will be ingested by a human being. Specific examples of such solvents include ethanol, propanol, butanol, butanone, acetone, methylene chloride, ethylacetate, etc.

The dosage forms produced via this invention will have an organic solvent content of less than 0.2 w/w % and more preferably less than 0.1 w/w % in which said organic solvent is selected from the group consisting of ethanol, propanol, butanol, acetone, butanone, and methylene chloride.

The following examples are being presented in order to further illustrate the invention. While they exemplify the production of dosage forms containing divalproex sodium, one skilled in the art could apply the teachings of these examples to other valproate compounds. These examples should not be construed in any manner as limiting the scope of the invention, or the claims attached hereto.

### EXAMPLE 1

Prior to use, divalproex sodium was reduced to small particles through a band (0.59 mm nominal mesh opening) with impact forward using a Fitzmill. The milled divalproex sodium is charged along with excipients (povidone K30, sodium starch glycolate, microcrystalline cellulose or dicalcium phosphate) into a Collette Gral 10 high shear mixer. The material is dry mixed for 5 min at low impeller speed. (200 rpm). The material then is wet massed with a 0.1 N citric acid solution as the granulation fluid at a high chopper speed (3000 rpm) and high impeller speed. (500 rpm) until granulation is complete. The material is dried in a fluid bed dryer to an LOD of not more than 2.0%. The dried granulation is milled through a band (0.84 mm nominal mesh opening) with knife forward using a Fitzmill. The milled material is added to a V-blender with silicon dioxide (Syloid 244) and blended until uniform. The resulted blend is compressed on a rotary tablet press into 720 mg tablets that contain 500 mg valproic acid equivalent or 1080 mg tablets that contain 750 mg valproic acid equivalent. The tablet compositions are given in Table 1.

TABLE 1

| Item | Ingredients | Code | mg/tab | % Load |
|---|---|---|---|---|
| 1 | Divalproex Sodium | 65476 | 538.8 | 74.8 |
| 2 | Sodium Starch Glycolate | 66851 | 28.8 | 4.0 |
| 3 | Povidone K30 | 57577 | 28.8 | 4.0 |
| 4 | Microcrystalline Cellulose, Avicel | 19615 | 100.8 | 14.0 |
| | PH101 | | | |
| 5 | Citric Acid, 0.1 N | 11694 | 1.2 | 0.2 |
| 6 | Silicon Dioxide (Syloid 244 FP) | 63337 | 21.6 | 3.0 |

### EXAMPLE

Prior to use, divalproex sodium was reduced to small particles through a band (20 mesh) with impact forward using a Fitzmill. The milled divalproex sodium is charged along with ith excipients (povidone K30, Prosolv 50 or 90) into a Collette Gral 10 high shear mixer. The material is dry mixed for 5 min at low impeller speed. (200 rpm). The material then is wet massed with a 20 to 30% PVP solution (w/w, in 0.1 N citric acid) as the granulation fluid at a high chopper speed (3000 rpm) and high impeller speed. (500 rpm) until granulation is complete. The material is dried in a fluid bed dryer to an LOD of not more than 2.0%. The dried granulation is milled through a band (16 mesh) with impact forward using a Fitzmill. The milled material is added to a V-blender with silicon dioxide (syloid 244) and blended until uniform. The resulted blend is compressed on a rotary tablet press into 670 to 720 mg tablets that contain 500 mg valproic acid equivalent or 1010 to 1080 mg tablets that contain 750 mg valproic acid equivalent. The tablet compositions are given in Table 2 and 3.

TABLE 2

| Item | Ingredients | Code | mg/tab | % Load |
|---|---|---|---|---|
| 1 | Divalproex Sodium | 65476 | 538.2 | 80 |
| 2 | Prosolv (silicified mycrocrystalline | N/A | 101 | 15 |
| | cellulose) | | | |
| 3 | Povidone K30 | 57577 | 10 | 1.5 |
| 4 | Povidone K30 (in solution) | 57577 | 10 | 1.5 |
| 5 | Citric Acid | 11694 | 0.07 | <0.01 |
| 6 | Silicon Dioxide (Syloid 244 FP) | 63337 | 13.5 | 2 |
| Total | | | 672.8 | 100 |

TABLE 3

| Item | Ingredients | Code | mg/tab | % Load |
|---|---|---|---|---|
| 1 | Divalproex Sodium | 65476 | 538.2 | 75 |
| 2 | Prosolv (silicified mycrocrystalline | N/A | 143.1 | 20 |
| | cellulose)s | | | |
| 3 | Povidone K30 | 57577 | 10.75 | 1.5 |
| 4 | Povidone K30 (solution) | 57577 | 10.75 | 1.5 |
| 5 | Citric Acid | 11694 | 0.07 | <0.01 |
| 6 | Silicon Dioxide (Syloid 244 FP) | 63337 | 14.5 | 2 |
| Total | | | 720 | 100 |

While there have been and shown and described what are the preferred embodiments of the invention, one skilled in the art will appreciate that various modifications in the formulations and processes can be made without departing from the scope of the invention as it is defined by the appended claims.

### EXAMPLE 3

This example illustrates the results of in vitro dissolution testing on a dosage form prepared using the methods described above in Example 1 and 2.

### In vitro dissolution test

In vitro dissolution rate of the tablets were compared with that of the reference, depakote, the uncoated marketed tablet, which contains the same amount of the active ingredient. USP apparatus II was used for testing. The test condition was: paddle speed=50 rpm; dissolution medium=900 ml 0.5M phosphate buffer at pH 7.5; temperature=37° C. Dissolution samples were analyzed by a TDX fluorescence-polarized immunoassay.

### Results and Discussions

In vitro dissolution profiles of the reference tablet and tablet from the current invention shown in FIG. 1 indicate that dissolution of the current invention is rapid, complete and equivalent to the reference (>90% in 20 minutes). Because divalproex sodium is a soluble, permeable and stable compound and known to have complete oral absorption, equivalent dissolution in vitro can result in equivalent in vivo absorption.

Acids that can be used in the current invention to control pH of granulation fluid include all pharmaceutically accepted organic (e.g. citric acid, tartaric acid) and inorganic acids (e.g. hydrochloric acid) Other ingredients include pharmaceutically acceptable excipients, including fillers, disintegrants, lubricants, glidants and binders.

[image]

### EXAMPLE 4

Tablets produced using the methods of Examples 1 and 2 were subjected to static headspace gas chromatography and analysed for the presence of methanol, ethanol and acetone.

### Static Headspace Gas Chromatography

As stated in the Ph. Eur. (2.2.28), “Static head-space gas chromatography is a technique particularly suitable for separating and determining volatile compounds present in solid or liquid samples. The method is based on the analysis of the vapour phase in equilibrium with the solid or liquid phase. The sample to be analysed is introduced into a container fitted with a suitable stopper and a valve-system which permits the passage of the carrier gas. The container is placed in a thermostatically controlled chamber at a temperature set according to the substance to be examined. The sample is held at this temperature long enough to allow equilibrium to be established between the solid or liquid phase and the vapour phase. The carrier gas is introduced into the container and, after the prescribed time, a suitable valve is opened so that the gas expands towards the chromatographic column taking the volatilised compounds with it.”

### Testing Instructions for the Determination of Ethanol, Acetone, and Methanol (or other organic) in Depakote 500 mg IR Tablets, by Static Headspace Gas Chromatography

### Residual Solvents

Examine by gas chromatography [Ph. Eur. (2.2.28)], using a head space injector and vials which can be crimp-sealed and which are compatible with the injector.

### Reference Solution

Dilute 110 mg of ethanol (R) in dimethylformamide (R) and make up to 50.0 mL with the same solvent=solution (1)

Dilute 50 mg of acetone (R) in dimethylformamide (R) and make up to 100.0 mL with the same solvent=solution (2)

Dilute 50 mg of methanol (R) in dimethylformamide (R) and make up to 100.0 mL with the same solvent=solution (3)

In a volumetric flask of 100 mL, introduce 25.0 mL of solution (1), 10.0 mL of solution (2), 10.0 mL of solution (3) and make up to volume with dimethylformamide (R).

In a vial which is compatible with the injection system, introduce 1.0 mL of the reference solution and crimp-seal the vial.

### Test Solution

Place 2 tablets, coarsely ground in a 50 mL glass vial with grinded stopper and add 20.0 mL of dimethylformamide (R). Shake 5 minutes on a magnetic agitator and 10 minutes with ultrasonic waves. Allow the solution to place and introduce 1.0 mL of the supernatant in the vial which is compatible with the injection system and crimp-seal the vial.

The reagents (R) are described in the Ph. Eur.

### Injection Sequence

1. Inject the reference solution 6 times, in a manner which brackets the test solution.

Inject the test solution.

### Chromatographic Conditions and Equipment

### Hewlett Packard 7694 Headspace GC

| | |
|---|---|
| Zone temperatures | |
| Oven - 90° C. | Shaker setting - Off |
| Loop - 110° C. | |
| Transfer line - 120° C. | Pressure - 20 psi |
| Timed events | |
| GC cycle time - 30 minutes | |
| Inject time - 0.5 minutes | Injection volume - 1 mL |
| Loop equilibration - 0.03 minute | |
| Loop fill time - 0.15 minutes | |
| Vial Pressurization time - 0.12 minutes | Pressure - 16.3 psi |
| Vial equilibration time - 15 minutes | |

### Hewlett Packard 5890 Series II Plus GC

Injection temp-200° C.

Injection type-Split; split flow: 50 mL/min

Oven program

110° C. for 8 minutes, ramp 20° C./min to 240° C. for 6 minutes; 2 minute oven equilibration

Detector temp-270° C.

Helium flow

inlet B-16 psi (3.7 mL/min)

Column: Chrompack Poraplot Q Plot FS (10 m×0.32 mm, 10 um film)

### Results

The following results were obtained:

| Solvent | Amt | |
|---|---|---|
| Methanol | <0.0351 | mg/tablet |
| Ethanol | <1.964 | mg/tablet |
| Acetone | <0.1249 | mg/tablet |

We claim:

1. A pharmaceutical dosage form comprising:
a) divalproex sodium;
b) optionally one or more pharmaceutical excipients, and;
c) an organic solvent content of less than 0.2 w/w %, based upon the total weight of the dosage form.

2. The pharmaceutical dosage form according to claim 1 in which said organic solvents are selected from the group consisting of ethanol, propanol, butanol, acetone, butanone, ethyl acetate, and methylene chloride.

3. A process for granulating divalproex sodium comprising:
a) contacting divalproex sodium with a sufficient quantity of a binding agent, optionally one or more additional pharmaceutical excipients, and a sufficient quantity of an aqueous solution having a pH of 5 or less, and
b) mixing said divalproex sodium with said binding agent, said optional excipients and said acidic solution for a sufficient period of time to produce a granulate.

4. The process according to claim 3 in which said binding agent is present in the quantity of from about 2-10 w/w % based upon the total weight of the divalproex sodium and the optional excipients.

5. The process according to claim 3 in which said aqueous solution is present in the quantity of from about 5-25 w/w %, based upon the total weight of the divalproex sodium, said bind binder and said optional excipients present.

6. The process according to claim 3 in which said aqueous solution is a 0.0001-0.1 normal solution of a food grade acid.

7. The process according to claim 3 in which said aqueous solution has a pH of 3 or less.

8. The process according to claim 3 in which said granulate is processed into a solid dosage form.

9. A process for producing solid dosage forms of divalproex sodium comprising:
a) contacting divalproex sodium with a sufficient quantity of a binding agent, optionally one or more pharmaceutical excipients, and a sufficient quantity of an aqueous solution having a pH of 5 or less;
b) mixing said divalproex sodium with said binding agent, said optional excipient and said acidic solution for a sufficient period of time to produce a granulate suitable for incorporation into a solid dosage form;
c) drying said granulate;
d) processing said granulate to a particle size of less than 44 mesh, and;
e) producing a solid dosage form by either compressing or encapsulating said granulate of step d).

10. The process according to claim 9 in which said binding agent is present in the quantity of from about 2-10 w/w % based upon the total weight of the valproic compound and optional excipients.

11. The process according to claim 9 in which said aqueous solution is present in the quantity of from about 5-25 w/w %, based upon the total weight of the divalproex sodium, the optional excipients, and the binder present.

12. The process according to claim 9 in which said aqueous solution is a 0.0001-0.1 normal solution of a food grade acid.

13. The process according to claim 9 in which said aqueous solution has a pH of 3 or less.

14. A pharmaceutical dosage form comprising:
a) a valproate compound;
b) optionally one or more pharmaceutical excipients, and;
c) an organic solvent content of less than 0.2 w/w %, based upon the total weight of the dosage form.

15. A process for granulating valproate compounds comprising:
a. contacting a valproate compound with a sufficient quantity of a binding agent, optionally one or more additional pharmaceutical excipients, and a sufficient quantity of an aqueous solution having a pH of 5 or less, and
b. mixing said valproate compound with said binding agent, said optional excipients and said acidic solution for a sufficient period of time to produce a granulate.

16. The process according to claim 15 in which said binding agent is present in the quantity of from about 2-10 w/w % based upon the total weight of the valproate compound and optional excipients.

17. The process according to claim 15 in which said aqueous solution is present in the quantity of from about 5-25 w/w %, based upon the total weight of the valproate compound, binder and optional excipients present.

18. The process according to claim 15 in which said aqueous solution is a 0.0001-0.1 normal solution of a food grade acid.

19. The process according to claim 15 in which said aqueous solution has a pH of 3 or less.

20. A process for producing solid dosage forms of valproate compounds comprising:
a. contacting a valproate compound with a sufficient quantity of a binding agent, optionally one or more pharmaceutical excipients, and a sufficient quantity of an aqueous solution having a pH of 5 or less;
b. mixing said valproate compound with said binding agent, said optional excipient and said acidic solution for a sufficient period of time to produce a granulate suitable for incorporation into a solid dosage form;
c. drying said granulate;
d. processing said granulate to a particle size of less than 44 mesh, and;
e. producing a solid dosage form by either compressing or encapsulating said granulate of step d).

21. The process according to claim 20 in which said binding agent is present in the quantity of from about 2-10 w/w % based upon the total weight of the valproate compound and the optional excipients.

22. The process according to claim 20 in which said aqueous solution is present in the quantity of from about 5-25 w/w %, based upon the total weight of the valproate compound, optional excipients, and binder present.

23. The process according to claim 20 in which said aqueous solution is a 0.0001-0.1 normal solution of a food grade acid.
